(19) United States
(12) Patent Application Publication
(10) Pub. No.: US 2003/0095891 A1
(43) Pub. Date: May 22, 2003

(54) Sterilizing apparatus and method

(76) Inventor: William O'Neal, Loxahatchee, FL (US)

Correspondence Address: J. Rodman Steele, Jr., Akerman, Senterfitt & Eidson, P.A., P.O. Box 3188, West Palm Beach, FL, 33402-3188, US

(21) Appl. No.: 09/990,044

(22) Filed: Nov. 20, 2001

(51) Int. Cl. A61L 2/07

(52) U.S. Cl. 422/26; 422/30; 422/108; 422/112; 422/292; 422/295; 422/298; 422/299; 422/305; 422/307

(57) ABSTRACT

A sterilizing apparatus and method in which a sanitizing device detects pressure of steam rather than temperature. The pressure is related to a desired temperature, but detection of pressure means that temperature sensors do not need to be exposed to the steam. In addition, the steam can be fully saturated, and sterilization can be obtained without the need for a drying cycle. The liquid does not need to be reused in the apparatus, ensuring that biofilm contamination of articles can be substantially reduced.

### BACKGROUND OF THE INVENTION

1. Field of the Invention

This invention relates generally to a sterilizing apparatus and method, and more particularly relates to an autoclave for sterilizing articles using pressurized steam.

2. Description of the Related Art

Hospitals, laboratories, physicians and dental offices must sterilize instruments for use in surgeries and other procedures. The use of autoclaves to conduct sterilization procedures is well known. In a typical autoclave, water that has been stored in a container is fed into a chamber in which articles and instruments have been placed, and is then heated to form steam. Various official codes and requirements exist for the sterilization of articles, which generally require sterilization at a certain temperature for a certain length of time. Once the articles have been held at the required temperature for a sufficient length of time, a drying and cooling cycle occurs, enabling subsequent reuse of the articles. During the drying and cooling cycle, the steam is allowed to condense. The condensate is collected in the container and reused for further sterilization cycles.

Problems arise with conventional autoclaves because various temperature sensors such as thermistor probes and control microelectronics must be provided in order to detect the temperature of the steam and to keep the temperature constant during the sterilization cycle. Such electronic circuitry may become damaged and degraded in the atmosphere of the autoclave, reducing reliability. In addition, due to the reliability problems with the temperature sensing components, conventional autoclaves tend to run sterilization cycles for a longer period of time than necessary, in order to compensate for possible inaccuracies in the temperature readings. These longer cycle times, together with the need for drying cycles, mean that it is not possible to quickly sterilize articles to allow for very rapid reuse, requiring users of autoclaves to keep extra inventory of instruments that may be needed.

A further problem with conventional autoclaves arises from the reuse of water condensed from previous sterilization cycles which contain biological 10 contaminants and waste matter. In addition, bacteria and other organisms contained in the biological contaminants can multiply while water is stored in the container for reuse. The contaminants and waste matter can be deposited on articles in subsequent sterilization cycles as a biofilm contaminant, which is undesirable, and may also be corrosive to that articles and/or to the autoclave components.

### SUMMARY OF THE INVENTION

One aspect of the invention provides an apparatus for sterilizing articles. The apparatus includes a chamber for receiving articles to be sterilized. A heater for heating liquid to form steam is also included. The apparatus also has a pressure sensor, which is in communication with the chamber, and a control mechanism. The pressure of steam inside the chamber can be sensed by the pressure sensor. The control mechanism can maintain the steam at a predetermined pressure until the end of a sterilization cycle.

When the correct pressure has been reached in the sterilization chamber, because the pressure and temperature are interrelated, it may be deduced that the correct temperature has also been reached. The heating element may then be cycled on and off during the sterilization cycle in order to maintain a constant temperature and pressure of steam. Once the timing element indicates that the end of the cycle has been reached, the steam can be vented from the sterilization chamber.

At least two pressure sensors may be provided. Each pressure sensor can be operable to select different predetermined pressures. Each pressure sensor may be a valve operable at a predetermined pressure.

Preferably, the chamber is capable of receiving a predetermined volume of liquid, such as water or another liquid capable of forming steam. The control mechanism preferably includes a timer. The timer may be operable by a user of the apparatus. A vent valve to vent the chamber at an end of the sterilization cycle can be included. A liquid collection reservoir can be connected to the vent valve to collect and condense the steam.

A temperature selection switch operable by a user of the apparatus may be included. The temperature selection switch can control the predetermined pressure. Where two or more pressure sensors are used, the temperature selection switch can control which of the pressure sensors is operated.

Another aspect of the invention relates to a method of sterilizing articles. The method includes providing a sterilizing apparatus which has a chamber, a heater, a pressure sensor and a control mechanism. Articles to be sterilized are placed in the chamber. The method also includes supplying liquid such as water or another liquid capable of forming steam to the sterilizing apparatus, heating the liquid to form steam, and sensing the pressure of steam inside the chamber. When a predetermined pressure of steam inside the chamber is attained, the control mechanism can be activated to retain the steam at the predetermined pressure of steam until a sterilization cycle is completed.

Preferably, the heater is cycled on and off during the sterilization cycle to maintain the predetermined pressure of steam. The method may further include the step of selecting the predetermined pressure of steam from at least two predetermined pressures. Preferably, the liquid is supplied to the apparatus at a predetermined volume, which enables an accurate association of pressure of steam inside the chamber with its temperature. The method preferably further includes venting the steam at the end of the sterilization cycle.

Yet a further aspect of the invention relates to a method of sterilizing articles. The method includes providing a sterilizing apparatus including a chamber, a heater, and a control mechanism. In addition, the method includes placing articles to be sterilized in the chamber, supplying liquid to the sterilizing apparatus, heating the liquid to form saturated or superheated steam, retaining the saturated steam in the chamber for a predetermined period of time at a predetermined pressure, and venting the chamber, whereby the articles are substantially dry at the end of a sterilization cycle.

The method may also include the step of retaining the articles in the chamber for a period of time at the end of a sterilization cycle to thereby cool the articles. The predetermined pressure may be selectable from at least two different predetermined pressures. The saturated steam may be heated while it is retained in the chamber in order to maintain the saturated steam at the predetermined pressure. The heating of the saturated steam may be intermittent during a sterilization cycle.

A further aspect of the present invention relates to a method of reducing biofilm contamination in a sterilizing apparatus. The method includes providing fresh liquid to the apparatus, heating the fresh liquid to form steam and retaining the steam in the apparatus for a predetermined period of time in order to sterilize articles placed in the apparatus. The method further includes venting the apparatus, condensing the steam to form waste liquid, and discharging the waste liquid from the apparatus, instead of reusing the liquid.

It is understood, however, that the invention is not limited to these arrangements.

### BRIEF DESCRIPTION OF THE DRAWINGS

There are shown in the drawing embodiments which are presently preferred, it being understood, however, that the invention is not limited to the precise arrangements and instrumentality shown, wherein:

FIG. 1 is a front perspective view of a sterilizing apparatus according to the invention;

FIG. 2 is a schematic view of operating components of the apparatus of FIG. 1;

FIG. 3 is a flowchart illustrating a method of operating the sterilizing apparatus according to the invention; and

FIG. 4 is a flowchart illustrating a method of reducing biofilm contamination in a sterilizing apparatus.

### DETAILED DESCRIPTION OF THE PREFERRED EMBODIMENTS

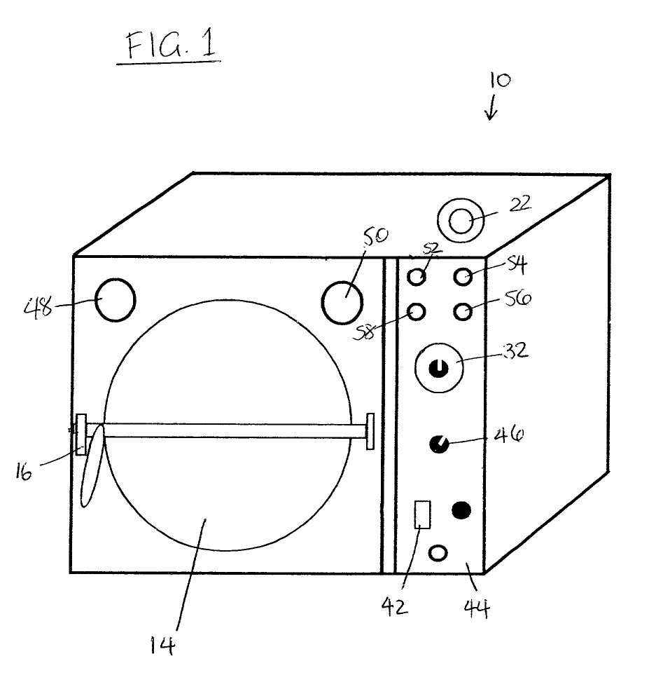

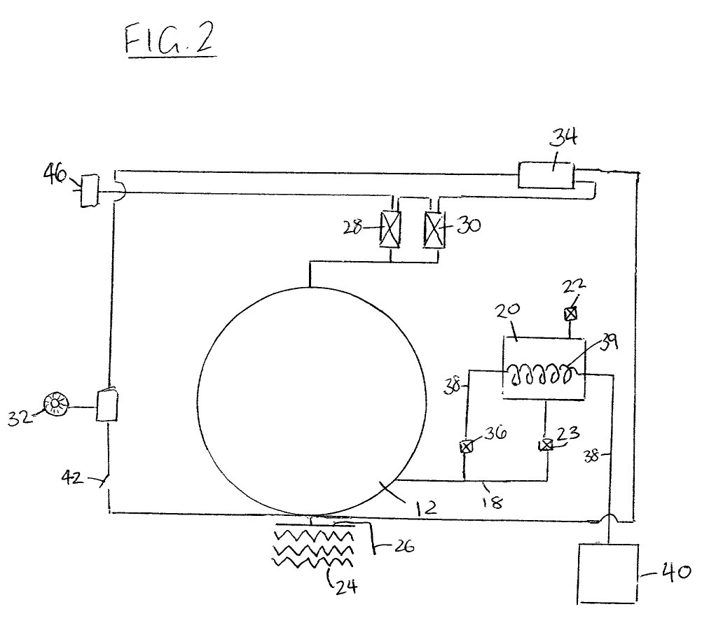

A sterilizing apparatus 10 according to the invention is shown in FIG. 1, with the operating components shown in FIG. 2. The apparatus 10 is suitable for use with any materials that are capable of being sterilized, such as surgical, dental and laboratory instruments. For example, the apparatus 10 can be used to sterilize straight or hinged stainless steel instruments, carbon steel instruments, heat resistant plastic items, glass slabs, beakers and stones, gauze and liquids. The apparatus 10 includes a sterilization chamber 12, to which access is permitted by a door 14. The door can have a security lock 16. One or more trays (not shown) may be provided to hold articles to be sterilized in appropriate positions within the chamber 12. The chamber 12, door 14 and trays can be made of any suitable material, and in one arrangement are made of metal that can resist corrosion by steam. In another arrangement, the chamber 12 can be made of heavy gauge electropolished stainless steel, and the door 14 can be made of cast aluminum.

A liquid supply conduit 18 supplies liquid, such as water or another liquid capable of forming steam, to the chamber 12. The liquid supply conduit can be connected to a liquid reservoir 20 having a first supply valve 22. A second supply valve 23 can be located in or near the liquid supply conduit 18, in order to control the amount of liquid supplied from the reservoir 20 to the chamber 12. The reservoir 20 and the liquid supply conduit 18 can be made of metal, such as stainless steel, although the invention is not limited in this regard. A heating element 24 can be in any location relative to the chamber, and in one embodiment is provided underneath the chamber 12. The heating element 24 may be any suitable material. For example, the heating element 24 can be copper coated nickel cadmium. The heating element 24 can be connected to any suitable power source via a lead 26. Any number of separate pressure valves 28 and 30 can be provided in communication with the sterilization chamber 12. The valves 28 and 30 can be set to operate at different pressures, such as 20 psi and 30 psi. It should be appreciated that these pressures are exemplary only and that other operating pressures and arrangements of valves may be used.

A timer 32 can be connected to the heating element 24 via a relay 34, and also to a vent valve 36. The timer 32 can be an analog electromechanical timer, although any suitable timer, including an electronic timer, may be used. The vent valve 36 can be located in the liquid supply conduit 18 and can be connected to a vent conduit 38. The vent conduit 38 can travel through the liquid supply reservoir 20, and may be coiled in the reservoir 20 to form a condensing coil 39 which provides heat exchange between steam in the vent conduit 38 and liquid in the reservoir 20. In one arrangement there is no fluid communication between the vent conduit 38 and the reservoir 20, so that the liquid in reservoir 20 cannot be contaminated with any waste products entrained in the steam vented through the vent conduit 38. The vent conduit 38 can be further connected to a waste collector 40, or alternatively to a drain (not shown).

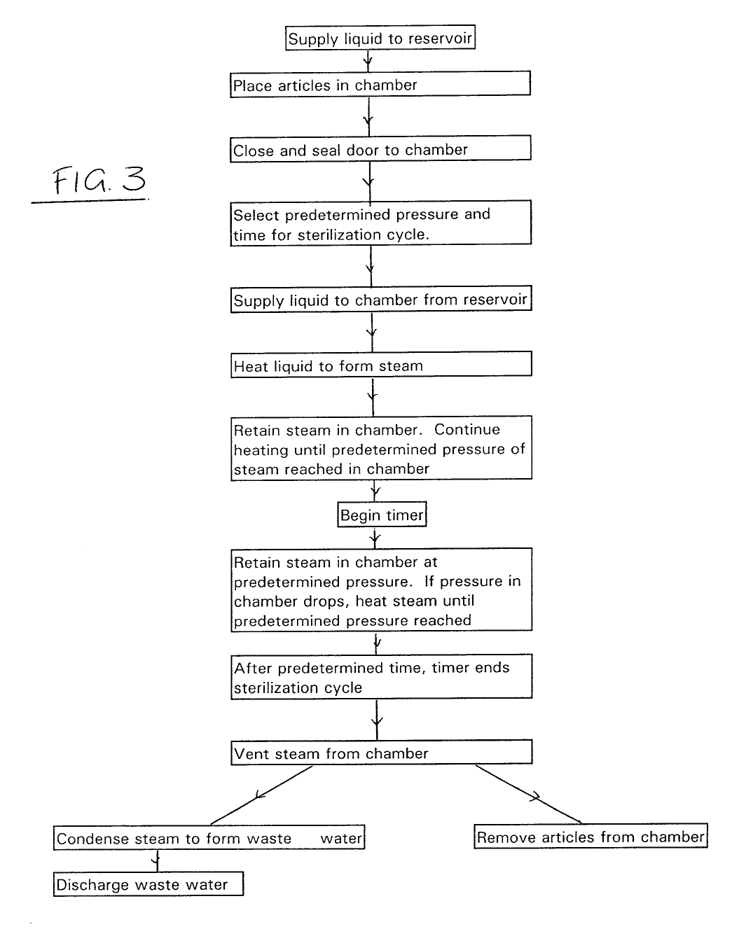

In operation, the liquid reservoir 20 can be filled via supply valve 22 with water or other liquid capable of forming steam, for example a mixture of water and one or more cleaning products. Referring to FIG. 3, the supply of liquid to the reservoir is illustrated as step 60. In step 62, the items to be sterilized can be placed in the chamber 12 on suitable trays or racks (not shown) that allow for the efficient circulation of steam therearound. When the door 14 is closed (step 64 in FIG. 3), it can be sealed shut by use of the security lock 16. A desired quantity of liquid can then be supplied to the chamber 12 from the liquid reservoir 20 via the liquid supply conduit 18 and the second supply valve 23. In one arrangement, it is preferred but not required that distilled water having less than 3 ppm of dissolved solids is used, so that impurities are not introduced into the chamber 12, and also to ensure that only minimal cleaning of the chamber is required. FIG. 3 illustrates the supply of liquid to the chamber as step 68.

The sterilization cycle can be initiated by operation of a switch 42 in a control panel 44 on the sterilizing apparatus 10. The desired temperature or pressure of the sterilizing cycle can be selected by means of a selection switch 46, and the cycle time can be selected by means of the timer 32. The selection of a predetermined pressure and time for the sterilization cycle is illustrated in FIG. 3 as step 66. The selection switch 46 may be marked with suitable temperatures for selection by a user, although the selection switch 46 is actually a pressure selection switch in that it can select one or more of the pressure valves 28 and 30 to detect the pressure of the steam in the chamber 12, and can deactivate any nonselected valve. In an alternative arrangement, the selection switch 46 may be marked with suitable pressures for selection by the user.

The apparatus may include a pressure indication gauge 48, a temperature indication gauge 50, a “power on” indicator light 52, a “heat on” indicator light 54, a “door open” indicator light 56, and a “reservoir low” indicator light 58. Other indicators or control switches may also be provided.

The heating element 24 can be used to heat the liquid in the chamber 12 to form steam, which is illustrated as step 70 in FIG. 3. Alternatively, the heating element 24 may be provided adjacent to the liquid supply conduit 18 or liquid reservoir 20 so that pre-heated steam may be provided to the chamber 12. Preheating the steam in a conduit or reservoir may be appropriate for a large sized chamber 12. The steam can be continually heated by the heating element 24 to increase the temperature of the steam until the desired temperature is reached. This is shown as step 72 in FIG. 3. By using a known volume of liquid from the reservoir 20, and because steam expands with temperature, once the pressure of the steam inside the apparatus 10 reaches a predetermined value, it is known that the steam has also reached a desired temperature using the well-known equation pressure×volume=temperature (pV=T). It has been found that in one embodiment of the sterilizing apparatus according to the present invention, 20 psi equates to a temperature of 250° F. and 30 psi equates to a temperature of 272° F. It should be noted that the invention in not limited in this regard.

It should be noted that the heating element 24 can remain on constantly until the desired pressure of steam has been reached. This arrangement ensures that the steam is fully saturated, and that no liquid droplets are included in the steam in the chamber 12, which allows sterilization to be achieved without the use of a drying cycle. Once the desired pressure has been reached, the timer 32 is started (step 74), and the timer 32, selected valve 28 or 30 and relay 34 can cycle the heating element 24 on and off in order to maintain a constant temperature and pressure in the sterilizing chamber 12. FIG. 3 illustrates the retention of steam in the chamber at a predetermined pressure as step 76.

As an alternative to the selection of the time by a user, preset sterilizing programs may be provided. In this arrangement, the programs can be controlled by a microprocessor (not shown), suitably shielded from the interior of the chamber 12. Examples of suitable programs are set out below.

### EXAMPLE 1

A program for unwrapped articles such as non-surgical instruments loose on a tray, open glass or metal canisters, and heat resistant rubber tubing which will not be used in any surgical application can be run at 270° F. for 6 minutes.

### EXAMPLE 2

A program for wrapped articles such as loosely wrapped individual instruments, multiple layers of instruments separated by fabric, instruments in paper bags, wrapped trays of loose instruments and heat-resistant rubber tubing can be run at 270° F. for 10 minutes.

### EXAMPLE 3

A program for packs such as common groups of surgical instruments in commercially-prepared packs, surgical instruments subject to prolonged storage, and surgical gloves wrapped for sterilization can be run at 250° F. for 30 minutes. This program is also suitable for any items, other than liquids, where 250° F. for 30 minutes is appropriate.

### EXAMPLE 4

A program for liquids, such as liquids or gels that could boil over or spill out of the container may be run at 250° F. for 30 minutes. At the end of the sterilizing cycle, venting is slowed to allow heat in the liquid to dissipate slowly and eliminate boil-overs. This program does not require a drying cycle.

At the end of the desired cycle time, the timer or program controller can deactivate the heating element 24 (step 78 in FIG. 3), and operate the vent valve 36 to vent the steam to the vent conduit 38 (step 80 in FIG. 3). The condensing coil 39 can cool the steam due to heat exchange with the liquid remaining in the reservoir 20 (step 82 in FIG. 3), and the waste liquid can be discharged to the waste collector 40 or to a drain (step 84 in FIG. 3). Where the steam has been maintained at a desired pressure, venting of the chamber 12 can be achieved quickly. If heavy gauge stainless steel is used to form the chamber 12, the chamber can have good heat retention characteristics. In such an arrangement, if items are left in the chamber 12 after venting has occurred, the heat retained by the chamber can dry the articles if there is any moisture thereon. In addition, the steam can be fully saturated or superheated so that there are generally no liquid droplets present in the steam, leaving the items in the chamber 12 dry so that often no drying cycle is needed. These features enable the sterilization cycle to be quicker than with known devices. Additionally, where there are no microelectronics or temperature sensors in communication with the chamber 12, the apparatus can be more reliable than known sterilizers because there are no sensitive components that may be degraded by the steam. The articles are removed from the chamber at the end of the cycle, which is illustrated as step 86 in FIG. 3.

The sterilizing apparatus 10 can use clean water or other liquid in the chamber 12 every time the sterilizing cycle is run, allowing for the possibility of ensuring that the instruments are clean as well as sterile. Such an arrangement allows for a substantial reduction in biofilm contamination of the articles because contaminants are not deposited on articles in subsequent sterilization cycles. Although in the illustrated embodiment, the liquid supply conduit 18 is used in part for both supply of liquid and venting steam, completely separate liquid supply and venting conduits may be used to ensure no contamination of clean water or other liquid that is to be used for a sterilizing cycle takes place. The stainless steel reservoir 20 and liquid supply conduit 18 resist growth of biological contaminants better than the prior art plastic reservoirs, and so a completely separate vent conduit is not necessary in many instances.

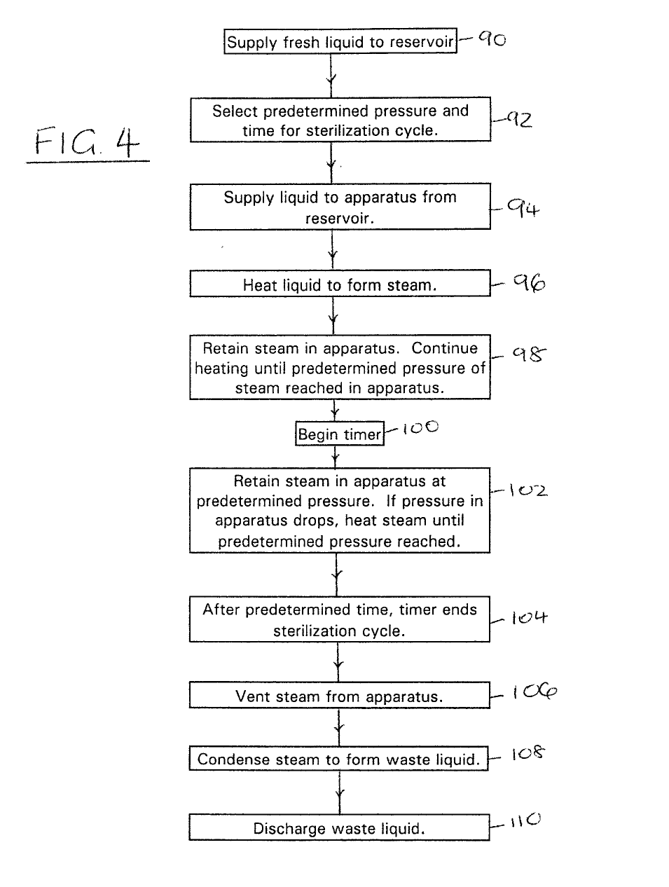

The reduction of biofilm contamination can occur on a continuous basis as the sterilization cycles are run, however, separate cleaning cycles can also be run to further aid in the reduction of contamination. Referring to FIG. 4, such a method includes the step 90 of providing fresh liquid to the reservoir. The predetermined pressure and sterilization time is set (step 92). Step 94 includes supplying liquid to the apparatus from the reservoir. Liquid is heated at 96 to form steam. Step 98 includes retaining the steam in the apparatus. The steam is continuously heated until the predetermined pressure of steam is reached in the apparatus. The timer is then started (100). Step 102 includes retaining steam in the apparatus at the predetermined pressure. If the pressure in the apparatus drops, the steam is heated until the predetermined pressure is reached again. The timer ends the sterilization cycle in step 104 after the predetermined time has elapsed. Step 106 includes venting steam from the apparatus, step 108 includes condensing the steam to form waste liquid, and final step 110 includes discharging the waste liquid.

It should be appreciated that the invention is not limited to any particular order of steps shown in the drawings and described in the specification. Additionally, it should be understood that the invention does not require all of the steps shown in the drawings and described in the specification.

Furthermore, it should be understood that the examples and embodiments described herein are for illustrative purposes only and that various modifications or changes in light thereof will be suggested to persons skilled in the art and are to be included within the spirit and purview of this application. The invention can take other specific forms without departing from the spirit or essential attributes thereof.

What is claimed is:

1. An apparatus for sterilizing articles, comprising:
a chamber for receiving articles to be sterilized;
a heater for heating liquid in said chamber to form steam;
a pressure sensor in communication with said chamber; and
a control mechanism, wherein the pressure of steam inside said chamber is sensed by said pressure sensor, and said control mechanism maintains the steam at a predetermined pressure until an end of a sterilization cycle.

2. The apparatus of claim 1, wherein said heater is adapted to be cycled on and off by said control mechanism during the sterilization cycle to maintain the predetermined pressure of steam.

3. The apparatus of claim 1, wherein at least two pressure sensors are provided, each said pressure sensor operable to select different predetermined pressures.

4. The apparatus of claim 1, wherein said pressure sensor is a valve operable at a predetermined pressure.

5. The apparatus of claim 1, wherein the chamber is capable of receiving a predetermined volume of liquid.

6. The apparatus of claim 1, wherein said control mechanism includes a timer.

7. The apparatus of claim 6, wherein said timer is operable by a user of the apparatus.

8. The apparatus of claim 1, further comprising a vent valve to vent said chamber at an end of the sterilization cycle.

9. The apparatus of claim 8, further comprising a liquid collection reservoir connected to said vent valve.

10. The apparatus of claim 1, further comprising a temperature selection switch operable by a user of the apparatus, said selection switch controlling said predetermined pressure.

11. A method of sterilizing articles comprising the steps of:
providing a sterilizing apparatus having a chamber, a heater, a pressure sensor and a control mechanism;
placing articles to be sterilized in the chamber;
supplying liquid to the apparatus
heating the liquid to form steam;
sensing the pressure of steam inside the chamber, wherein when a predetermined pressure of steam inside the chamber is attained, the control mechanism is activated to retain the steam at the predetermined pressure of steam until a sterilization cycle is completed.

12. The method of claim 11, wherein the heater is cycled on and off during the sterilization cycle to maintain the predetermined pressure of steam.

13. The method of claim 11, further comprising the step of selecting the predetermined pressure of steam from at least two predetermined pressures.

14. The method of claim 11, wherein the liquid is supplied to the apparatus at a predetermined volume.

15. The method of claim 11, further comprising venting the steam at the end of the sterilization cycle.

16. A method of sterilizing articles, comprising the steps of:
providing a sterilizing apparatus having a chamber, a heater, and a control mechanism;
placing articles to be sterilized in the chamber;
supplying liquid to the apparatus;
heating the liquid to form saturated steam;
retaining the saturated steam in the chamber for a predetermined period of time at a predetermined pressure; and
venting the chamber, whereby the articles are substantially dry at the end of a sterilization cycle.

17. The method of claim 16, further comprising the step of retaining the articles in the chamber for a period of time at the end of a sterilization cycle to cool the articles.

18. The method of claim 16, wherein the predetermined pressure is selectable from at least two different predetermined pressures.

19. The method of claim 16, wherein a predetermined volume of liquid is heated to form the steam.

20. The method of claim 16, wherein the saturated steam is heated while it is retained in the chamber in order to maintain the saturated steam at the predetermined pressure.

21. The method of claim 20, wherein the heating of the saturated steam is intermittent during a sterilization cycle.

22. A method of reducing biofilm contamination in a sterilizing apparatus, comprising the steps of:
providing fresh liquid to the apparatus;
heating the fresh liquid to form steam;
retaining the steam in the apparatus for a predetermined period of time in order to sterilize articles placed in the apparatus;
venting the apparatus;
condensing the steam to form waste liquid; and
discharging the waste liquid.

23. The method of claim 22, wherein the steam is retained in the apparatus at a predetermined pressure.

24. The method of claim 22, wherein a predetermined volume of fresh liquid is heated to form the steam.

25. The method of claim 22, wherein the fresh liquid and the waste liquid are kept separate from one another.

26. The method of claim 22, wherein the fresh liquid is heated to form saturated steam.
